(19) United States
(12) Patent Application Publication
(10) Pub. No.: US 2025/0288308 A1
(43) Pub. Date: Sep. 18, 2025

(54) SELF-FOCUSING MULTI-SPARK SHOCK WAVE GENERATOR FOR LITHOTRIPSY AND METHODS OF USING SAME

(71) Applicant: Duke University, Durham, NC (US)

(72) Inventors: Pei Zhong, Durham, NC (US); Georgy N. Sankin, Durham, NC (US); Obed Isaac, Durham, NC (US); Zheng Fang, Durham, NC (US)

(21) Appl. No.: 19/074,946

(22) Filed: Mar. 10, 2025

Related U.S. Application Data
(60) Provisional application No. 63/564,650, filed on Mar. 13, 2024.

(51) Int. Cl. A61B 17/22; A61B 17/00

(52) U.S. Cl. CPC A61B 17/22022; A61B 2017/00929; A61B 2017/22025; A61B 2017/22028

(57) ABSTRACT

A shock wave generator comprising a base, a plurality of transducers positioned on the base, a control assembly electrically coupled to the plurality of transducers, and a first chamber with a first fluid. The first chamber is at least partially defined by the base. The shock wave generator further comprises a second chamber with a second fluid, a membrane positioned between the first chamber and the second chamber, and a circulation assembly fluidly coupled to the first chamber. The circulation assembly includes a pump that circulates the first fluid, a chiller that controls the temperature of the first fluid, and a degasser that removes bubbles from the first fluid.

### RELATED APPLICATIONS

This application claims priority to and the benefit of U.S. Provisional Patent Application No. 63/564,650 filed Mar. 13, 2024, which is incorporated herein by reference in its entirety for all purposes.

### FEDERAL FUNDING

This invention was made with Government support under Federal Grant Nos. DF052985 and 1R01EB025205 awarded by the NIH and by Federal Grant No. R37DF052985-20 awarded by the National Institute of Diabetes & Digestive & Kidney Diseases (NIH/NIDDK). The Federal Government has certain rights to this invention.

### BACKGROUND

Kidney stone disease (or urolithiasis) affects 5-15% of the population worldwide. In the United States, shock wave lithotripsy (SWL) is still used as the first line therapy for about 50% of the stone patients. During clinical SWL, however, elevated dose delivered at fast rate is often preferred, in association with the high pressure and pulse energy generated by the contemporary lithotripters, which increases the risk of renal injury, i.e., hemorrhage and perinephric hematomas. It is therefore highly desirable to reduce the total number of high energy shock waves delivered to the patient to minimize the risk of tissue injury while maintaining successful stone comminution in SWL.

Two primary factors have been identified that contribute to the elevated shock wave exposure during SWL: 1) patient's respiratory motion and 2) residual fragment dispersion. Kidney stones may move by 10-40 mm and ureteral stones by 7-10 mm in patients under sedation during SWL, leading to about 40% of the lithotripter-generated shock waves (LSWs) missing the target stones. In addition, following the initial breakup, residual fragments may spread out inside the renal collecting system to an area exceeding the fragmentation zone of the lithotripter, rendering the ensuing shock waves less effective.

The respiration trajectory of the kidney is roughly aligned in parallel to the spine along the head-to-toe direction of the patient. In contrast, the spreading of residual fragments will depend on the anatomic features of the kidney (i.e., renal calyces and pelvis), or the orientation of the ureter where the stone resides. Matching the pressure or energy distribution with these anatomic features or respiratory motion patterns in stone patients during SWL are difficult using contemporary clinical lithotripters because they can only generate axisymmetric pressure fields along the LSW propagation direction. On the other hand, it is conceivable that an optimized distribution of the shock wave energy to maximally match with the stone or fragments trajectory during SWL will improve treatment efficiency while minimizing tissue injury.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Embodiments of the present disclosure include a shock wave generator comprising a base, a plurality of transducers positioned on the base, a control assembly electrically coupled to the plurality of transducers, and a first chamber with a first fluid. The first chamber is at least partially defined by the base. The shock wave generator further comprises a second chamber with a second fluid, a membrane positioned between the first chamber and the second chamber; and a circulation assembly fluidly coupled to the first chamber. The circulation assembly includes a pump that circulates the first fluid, a chiller that controls the temperature of the first fluid, and a degasser that removes bubbles from the first fluid.

In some embodiments, a first portion of the plurality of transducers are energized by the control assembly in a first configuration to generate a first beam shape; and a second portion of the plurality of transducers are energized by the control assembly in a second configuration to generate a second beam shape.

In some embodiments, wherein the first beam shape is circular, and the second beam shape is elongated.

In some embodiments, a third portion of the plurality of transducers are energized in coordination with the adjustment in energization and inactivation of the first and second portions of the plurality of the transducers by the control assembly to change either the size of the circular beam or the shape, size and orientation of the elongated beam.

In some embodiments, a first portion of the plurality of transducers are energized by the control assembly in a first configuration to generate a first beam size; and a second portion of the plurality of transducers are energized by the control assembly in a second configuration to generate a second beam size.

In some embodiments, a first portion of the plurality of transducers are energized by the control assembly in a first configuration to generate a first beam shape in a first orientation; and a second portion of the plurality of transducers are energized by the control assembly in a second configuration to generate the first beam shape in a second orientation.

In some embodiments, a first portion of the plurality of transducers are energized by the control assembly in a first configuration to generate a first beam shape with a first pressure distribution; and a second portion of the plurality of transducers are energized by the control assembly in a second configuration to generate the first beam shape with a second pressure distribution.

In some embodiments, each of the plurality of transducers is independently energized by the control assembly.

In some embodiments, the control assembly includes a spark gap with a first contact, a second contact, and a gas positioned between the first contact and the second contact; wherein the spark gap is in a closed configuration when the gas is at a first pressure and the spark gap is in an open configuration when the gas is at a second pressure, higher than the first pressure.

In some embodiments, the gas is nitrogen and the first pressure is 1 bar and the second pressure is 7 bar.

In some embodiments, the control assembly includes a plurality of solid-state transducers.

In some embodiments, the plurality of transducers comprises at least 30 transducers.

In some embodiments, a first transducer of the plurality of transducers includes a plurality of pins; wherein the plurality of pins comprises at least 105 pins.

In some embodiments, the shock wave generator further comprises a ground electrode positioned in the first chamber; wherein the ground electrode is spherical shape and includes a grid pattern with a plurality of openings.

In some embodiments, a first transducer of the plurality of transducers includes an electrode with a ring, a ground electrode, and an insulator positioned between the electrode and the ground electrode; wherein the ring forms at least part of an outer circumferential surface of the electrode.

In some embodiments, the shock wave generator further comprises a reservoir with the first fluid in fluid communication with the first chamber; and wherein the second chamber is at least partially defined by a bellow.

In some embodiments, the membrane is a spherical plastic membrane with a hydrophilic coating.

In some embodiments, the first fluid is saline and the second fluid is water.

In some embodiments, the base is spherical and includes a plurality of bores that receive the plurality of transducers.

In some embodiments, the circulation assembly includes a first inlet, a second inlet, and first outlet fluidly coupled to the first chamber; wherein the first inlet is positioned opposite the second inlet; and wherein the first inlet and the second inlet direct the first fluid into the first chamber with a radial and circumferential direction.

The present disclosure provides a steerable and adjustable focusing electrohydraulic (SAFE) shock wave generator for flexible lithotripter field control utilizing a re-configurable multi-spark transducer array. Presented herein are the design principle, preliminary tests of acoustic field characterization and in vitro stone comminution, demonstrating several unique advantages of this new shock wave source that may provide flexible beam forming and stone targeting capability during clinical SWL procedures.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1A-1C illustrates (A) a model of a 45-pin transducers; (B) a photo of the 45-pin transducer on the SAFE; and (C) illustration of wave propagated from the SAFE and focus at kidney stones, with two side sections in shaded areas disconnected, leading to a focal zone elongation in the side-section direction.

FIG. 2A-2D illustrates schematic views of the transducers distribution on the spherical carrier and resulting pressure contour in the focal plane in (A) and (C) Case A, and (B) and (D) Case B.

FIG. 3A-3B illustrates (A) a photo of the stone holder with the illustration of stone phantom location; and (B) a diagram of the experimental setup for stone fragmentation test.

FIG. 4A-4B illustrates peak positive pressure (p+) and peak negative pressure (p−) distribution comparison on (A) focal plane (x- and y-axis); and (B) along wave propagation path (z-axis) between Case A and Case B.

FIG. 5A-5D illustrates pressure waveforms measured at focal point when input voltage U=15 kV and 20 kV in (A) and (B) Case A, and (C) and (D) Case B.

FIG. 6A-6C illustrates (A) Stone comminution (n=5) of fragments smaller than 2 mm (left) and 2.8 mm (right) after 150 pulses of shock wave treatment by the SAFE in case A (blue) and case B (red). Indicated p-values smaller than 0.05 for each group of results. Stone fragments after 150 pulses of shock wave treatment by the SAFE in both (B) Case A and (C) Case B.

FIG. 7 illustrates a schematic showing the working of a shock wave generator, with a stone positioned at the focal point.

FIG. 8A-8C illustrates results of a simple linear superposition simulation of the acoustic waves interacting on the focal plane for three different configurations of the multi-spark unit. The normalized pressures on the focal plane shown for each of these three scenarios as a contour plot. The elongated beam has an enhanced zone of higher pressure.

FIG. 9 illustrates a transducer with 45 pins (left) and one with 105 pins (right). For scale, the outer diameter of the transducer body is 18 mm.

FIG. 10 illustrates a sample case showing reduction in peak pressure with every shot if the pump is not turned on.

FIG. 11 are photographs showing the silicone rubber being used as the membrane.

FIG. 12 is a schematic of a shock wave generator including a housing and coupling unit design. A concave membrane reduces the pumping volume, and inflatable bellows couple the shock wave to the human body.

FIG. 13 illustrates peak pressure before and after 10,000 shots; measured at the focus, for two different voltages. 105 pin bowl 42 transducers, mounted on a different housing unit and power supply.

FIG. 14 illustrates a pressure scan at the focal plane for 1% saline, 105 pin 36 transducer configuration, with the ground electrode at 12.5 mm from the face of the transducers.

FIG. 15 illustrates a schematic drawing of the stone holders in use for the experiments.

FIG. 16A-16C illustrate device arrangements for generating focused shock waves (A) a spherical carrier with an array of transducers, (B) a flat array of transducers in conjunction with an acoustic lens, and (C) an array of transducers distributed on a cylindrical surface in conjunction with a paraboloid reflector.

FIG. 17A-17B illustrates different combinations of transducers that can produce either an elongated or an axisymmetric pulse at the focal plane. An elongated beam that can be steered to align with the kidney or the head-to-toe respiration movement direction.

FIG. 18A-18D illustrates the principle for producing a steerable and non-axisymmetric pressure distribution to match the direction of stone movement or distribution of stone fragments in vivo. By selectively activating certain transducer groups (A, B), the shape of the beam can be varied (C, D).

FIG. 19A-19B illustrates options for the choice of a ground electrode. The wire geometry facilitates low inductance L. It could be closed cell clusters of any shape such as concentric rings (A), or polygonal cells (B), to facilitate acoustic transmission. The electrode has a curvilinear shape to maintain a constant gap between positive and negative electrodes.

FIG. 20 is a schematic of a circulation assembly including a pumping circuit for the saline solution and a Hydrophilic P100 (Joninn, Denmark) coating to facilitate bubble removal. Coolant flow circulation can be with either a center nozzle (inlet/outlet), and/or two 45-degree nozzles (inlets, outlets).

FIG. 21 illustrates a shock wave generator including a first portion of the transducers energized in a first configuration to generate a first beam shape (circular) and a second portion of the transducers energized in a second configuration to generate a second beam shape (elongated).

FIG. 22 is a schematic of a shock wave generator including a control assembly and a circulation assembly.

FIG. 23 is a cross-sectional view of a portion of a shock wave generator.

FIG. 24 is a schematic for an experimental setup for pressure data.

FIG. 25 is a graph of pressure versus distance from focus for the setup of FIG. 24.

FIG. 26 is a graph of pressure versus run number with saline circulation.

FIG. 27 is a schematic of a stone comminution setup.

FIG. 28 is a graph of stone comminution results.

FIG. 29 is a graph of stone comminution results.

FIG. 30 is photographs of stone comminution results.

FIG. 31 is a graph of pressure versus time illustrating lifespan of the electrode after 60,000 pulses.

FIG. 32 is a schematic of a shock wave generator including a control assembly with a spark gap.

FIG. 33 is a schematic of the spark gap of FIG. 32.

FIG. 34 is a perspective cross-sectional view of an electrode.

FIG. 35 is an exploded view of the electrode of FIG. 34.

FIG. 36 is a perspective cross-sectional view of a shock wave generator, with some portions removed for clarity.

FIG. 37 is an exploded view of the shock wave generator of FIG. 36.

### DETAILED DESCRIPTION

The present disclosure provides systems and methods relating to shock wave generators or lithotripters.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

“About” is used to provide flexibility to a numerical range endpoint by providing that a given value may be “slightly above” or “slightly below” the endpoint without affecting the desired result.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

The systems described herein can be implemented in hardware, software, firmware, or combinations of hardware, software and/or firmware. In some examples, the systems described in this specification may be implemented using a non-transitory computer readable medium storing computer executable instructions that when executed by one or more processors of a computer cause the computer to perform operations. Computer readable media suitable for implementing the systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system described in this specification may be located on a single device or computing platform or may be distributed across multiple devices or computing platforms.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. INITIAL DESIGN: MATERIALS AND METHODS

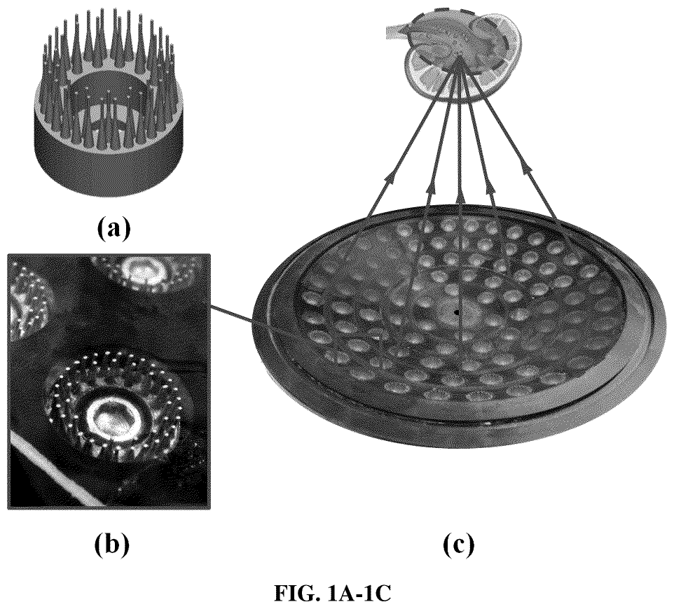

Design Concept. In the SAFE shock wave generator, 90 individual electrohydraulic transducers are mounted on a spherical carrier made of dielectric material. Each transducer consists of 45 3D-printed titanium electrodes embedded in epoxy resin and each polished to yield a tip diameter of 0.3 mm. The transducers are arranged in 5 concentric rings divided into 6 sectors (FIG. 1).

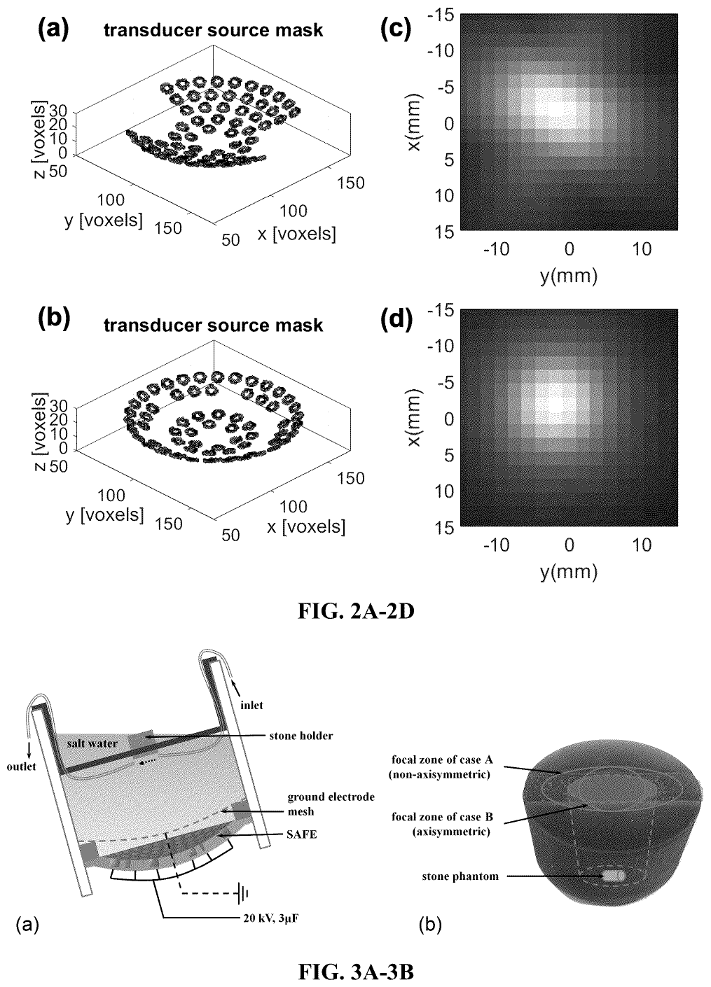

In linear acoustics, the beam width is inversely proportional to the aperture diameter of the acoustic source. Therefore, for a given focal length, by reducing the active section dimension of the shock wave generator, for example along a 45-degree angle with respect to the y-axis in FIG. 2, we can effectively increase the corresponding beam width in a particular direction. This design strategy is used to transform an axisymmetric pressure field into a non-axisymmetric, elongated focal zone (FIG. 2).

The numerical simulations throughout this pilot study were carried out using an open-source toolbox k-Wave in time-domain under linear wave propagation assumption. While the peak pressure may be underestimated, the linear wave propagation model is anticipated to qualitatively capture the focal pressure distribution for the purpose of comparison between the axisymmetric vs. elongated pressure fields. In the simulation, the voxel size was 1.875 mm, whereas the time step size was 0.156 microseconds.

Experiments were conducted using two different beam configurations, designed for employing the same total number of activated pins (Nₚ) yet with different transducer distributions in the SAFE shock wave generator. To produce a non-axisymmetric or elongated beam (Case A), 12 transducers on the two opposite sectors (12×2=24) were disconnected (FIG. 1C). In contrast, to produce an axisymmetric beam (Case B), all 4 transducers in the third ring for each of the six sectors (6×4=24) were disconnected (see FIG. 2). Hence, Nₜ=90−24=66 transducers were activated in both case A and case B. If we assume that the total input electric energy [Eₜₒₜₐₗ=CU²/2=0.5×3 μF×(15 kV)²=0.34 kJ] was evenly distributed to Nₚ activated pins, the electric energy delivered to each activated pin (Eₚ) can then be calculated by:

$${E}_{p} = \frac{{E}_{total}}{{N}_{p}} = \frac{\frac{1}{2} C {U}^{2}}{{N}_{p}} = 0.11 J \qquad ( 1 )$$

where Nₚ=Nₜ×45=2970, C=3 μF is the capacitance, and U is the charging voltage.

In summary, conventional clinical shock wave lithotripters produce an axisymmetric acoustic field without accounting for the anatomic features of the kidney or respiratory motion of the patient. As disclosed herein, a steerable and adjustable focusing electrohydraulic (SAFE) shock wave generator is configured to vary the beam size and shape.

Acoustic Field Characterization The SAFE shock wave generator, installed at the bottom of a cylindrical acrylic water tank (Ø254×H305 mm), was triggered by a digital delay pulse generator (BNC Model 555, Berkley Nucleonics). A metal mesh with grid size 12.5×12.5 mm was mounted above the surface of the titanium electrodes and grounded. The tank was filled with electrolyte (1% sodium chloride in water) to ensure synchronization of the spark discharges from all transducer tips. A fiber optic probe hydrophone (FOPH 500, RP Acoustics, Leutenbach, Germany) mounted on a computer-controlled 3D translational stage (VXM-2 step motors with BiSlide-M02 lead screw, Velmex, Bloomfield, NY) was used for scanning and pressure measurements in the shock wave focal plane.

The pressure measurements were performed at U=15 kV for beam configuration comparison. In addition, pressure was measured at the beam focus under U=20 kV, which was used for stone comminution experiments. The pressure measurements were repeated at least three times at each point.

Stone Fragmentation Assessment. Stone fragmentation experiments were performed using cylindrical soft BegoStone (5:2 powder to water mixing ratio) of Ø6×H6 mm in size and 0.33 g in weight. Stone phantoms were soaked in water for more than 30 minutes before the fragmentation test. As shown in FIG. 3, a polyurethane rubber stone holder (Ø48 mm outer diameter, 30 mm height) in elliptical shape (long axis: 24 mm, and short axis: 12 mm) was used to approximate the anatomic geometry of the renal pelvis while allowing residual stone fragments to be dispersed laterally during SWL.

A water circulation system was constructed to facilitate removal of bubble remnants accumulated underneath the stone holder during SWL. The pre-soaked stone sample was placed inside the polyurethane holder and aligned with the focus of the SAFE shock wave generator. After treatment with 150 pulses produced at 20 kV, stone fragments were collected and air dried overnight. Afterwards, fragments were filtered through 2 mm and 2.8 mm grid sieves (W. S. Tyler, Mentor, OH), and weighted to calculate the stone comminution efficiency based on the percentage of the residual fragments over the original stone weight. Six stones were treated for each case. Data were post-processed in Excel (Microsoft, Redmond, WA) and presented in bar chart with mean, standard deviation, and p-value.

In summary, 90 electrohydraulic transducers are mounted concentrically on a spherical basin with adjustable connection to individual transducers. Each transducer consists of 45 3D-printed titanium microelectrodes embedded in epoxy with a tip diameter of 0.3 mm. All the transducers are arranged in 5 concentric rings and sub-divided into 6 sectors.

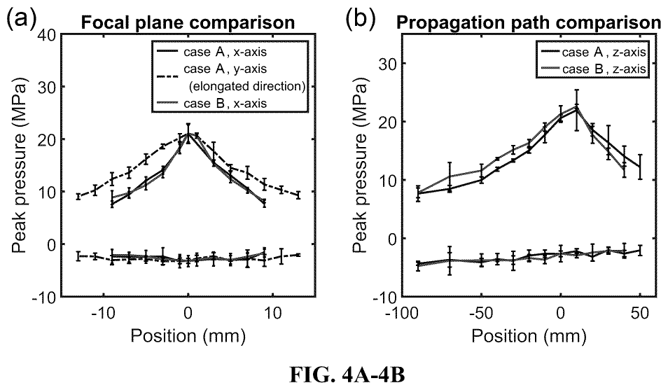

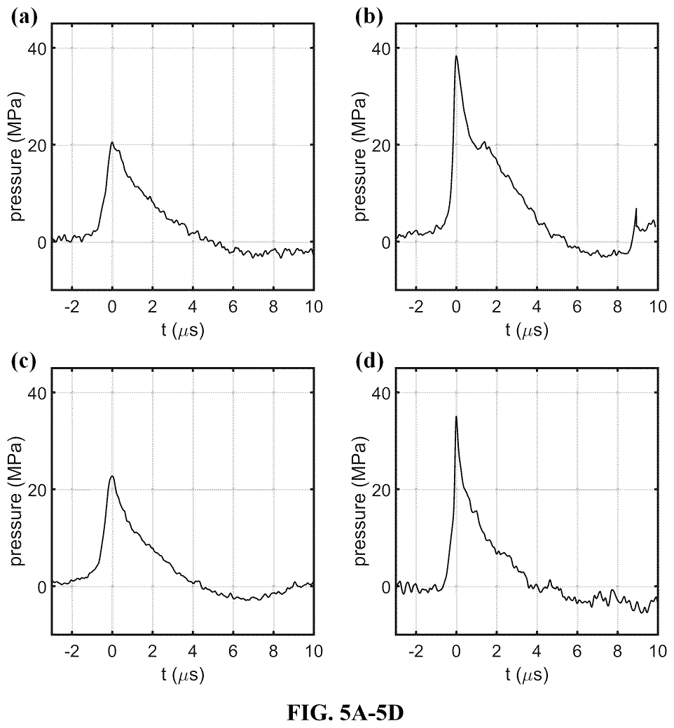

Results and Discussion. FIG. 4A shows the peak pressure (p+) distribution along two orthogonal directions in the focal plane (z=0 mm). The −6 dB focal width, estimated by the full width at half maximum using a Gaussian curve fitting for p+, was found to be 22.7 mm (along the side-section direction) by 15.1 mm (in the orthogonal direction) for the elongated beam. In comparison, the −6 dB focal width of the axisymmetric focal zone was about 14.8 mm in both directions. Moreover, the pressure distributions along the z-axis were found to be comparable between the two configurations (FIG. 4B). The slope of pressure change post-focally (i.e., z≥0) is steeper than its counterpart pre-focally (i.e., z≤0). These results are summarized in Table I. In comparison, based on linear wave model simulation, the −6 dB focal width is 18.5 mm along the side-section direction (Case A, non-axisymmetric, FIG. 2) by 11.5 mm. In contrast, the −6 dB focal width of the axisymmetric focal zone (Case B, axisymmetric, FIG. 2) is 13.0 mm along both directions. In general, the trends in the focal width change of the shock wave generator between experimental measurements and model simulations are similar, with an average discrepancy about 20%. These discrepancies are likely to be reduced when the nonlinear wave propagation is included in the future modeling work.

As the input voltage increases from 15 kV to 20 kV (with the corresponding total input electric energy varying from 338 J to 600 J), the value of p+ increases from 21.1±1.1 MPa to 33.7±4.1 MPa for the elongated beam. Similarly, p+ increases from 21.8±0.8 MPa to 36.2±0.7 MPa for the axisymmetric beam. At 20 kV, the rise time of the shock wavefront is in the range of 0.2 μs to 0.3 μs (Table II). These acoustic field parameters of the SAFE shock wave generator are comparable to the corresponding values in an HM3 lithotripter, except the longer rise time of the shock front (see Table II). In comparison to the HM3, the SAFE shock wave generator has the unique advantage of transforming the axisymmetric acoustic field in the lithotripter focal plane to an elongated (oval shape) pressure field that can better match with the anatomical features of the kidney and/or the trajectory of respiratory motion of the patients during SWL. More importantly, the SAFE shock wave generator has the potential of flexible control of the lithotripter focal beam size, shape, and orientations.

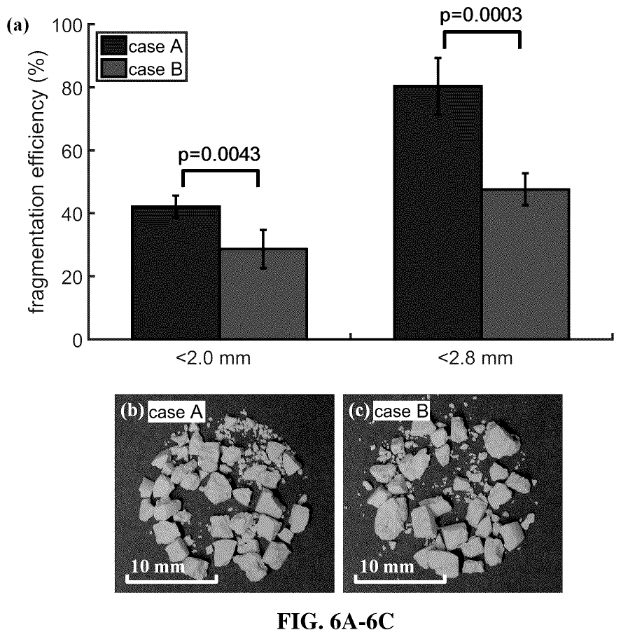

A total of 12 stones were treated. However, one outlier in each group was detected and removed, resulting in a final sample size of n=5. For the elongated beam, stone fragmentation rate for fragments less than 2.0 mm was 42.2±3.5% compared to 28.6±6.1% for the axisymmetric beam. For stone fragments smaller than 2.8 mm, stone comminution for the elongated beam (Case A: 80%±9%) is greater than that of the axisymmetric beam (Case B: 47.7%±5.1%). Two-tail t-test of the data from the two groups show p-values of 0.0043 (<2 mm) and 0.0003 for (<2.8 mm), respectively, indicating statistically significant difference in stone fragmentation produced by the two configurations of the SAFE. Graphically, FIGS. 6B and 6C display the stone fragments after SWL treatment. Stones treated by the elongated beam have more fragments and smaller sizes compared to those produced by the axisymmetric beam. These results suggest that higher stone comminution efficiency may be produced by adjusting the beam size and shape to better match with the target stone/fragments trajectory during clinical SWL procedures.

In summary, by changing the connections of individual transducers, the focused pressure field produced by the transducer array can be either axisymmetric with a −6 dB focal width of 14.8 mm in diameter, or non-axisymmetric with a long axis of 22.7 mm and a short axis of 15.1 mm. The elongated beam produces a peak positive pressure of 33.7±4.1 MPa and comminution efficiency of 42.2±3.5%, compared to 36.2±0.7 MPa and 28.6±6.1% for axisymmetric beam after 150 pulses at 20 kV.

Conclusion. A steerable and adjustable focusing electrohydraulic (SAFE) shock wave generator provides beam-forming flexibility in SWL, which allows us to better match the acoustic field of the lithotripter with anatomic features and spreading of residual fragments in stone patients. Improved stone comminution efficiency has been demonstrated in an elliptical stone holder. The SAFE shock wave generator can produce an elongated non-axisymmetric pressure field with higher stone comminution efficiency. The SAFE shock wave generator may provide a flexible and versatile design to achieve accurate, stable, and safe lithotripsy for kidney stone treatment.

TABLE I
Characteristics comparison of the SAFE in different cases.

| | | Transducer | Experiment | Experiment | Experiment | Simulation | Simulation |
|---|---|---|---|---|---|---|---|
| Case | Nₜ | geometry | p+(MPa) | FWₓ (mm) | FW_{y} (mm) | FWₓ (mm) | FW_{y} (mm) |
| A | 66 | non- | 21.1 | 15.1 | 22.7 | 11.5 | 18.5 |
| | | axisymmetric | | | | | |
| B | 66 | axisymmetric | 21.8 | 14.8 | 14.8 | 13.0 | 13.0 |

Note:
Nₜ is the number of activated transducers.

TABLE II
Comparison of the acoustic fields and stone comminution (SC) efficiency produced by the SAFE and Dornier HM3.

| | | Working | Working | | | | | | | | Stone |
|---|---|---|---|---|---|---|---|---|---|---|---|
| | | voltage | capacity | | Eₚ | p₊ | p₋ | tᵣ | t₊ | t₋ | comminution |
| | | U(kV) | C(μF) | Nₜ | (J/pin) | (MPa) | (MPa) | (μs) | (μs) | (μs) | (<2 mm) |
| SAFE | Case A | 20 | 3 | 66 | 0.202 | 33.7 ± 4.1 | −4.3 | 0.24 | ~5.5 | ~6.0 | 42% (150 pulses) |
| | Case B | | | 66 | 0.202 | 36.2 ± 0.7 | −5.5 | 0.31 | ~4.5 | ~7.0 | 29% (150 pulses) |
| HM3 | | 20 | 0.08 | 1 | 16 | 48.9 | −8.0 | <0.030 | 1~2 | 4~6 | <30% (150 pulses |
| | | | | | | | | | | | in the |
| | | | | | | | | | | | membrane holder) |

### 3. IMPROVEMENTS TO SHOCK WAVE GENERATOR

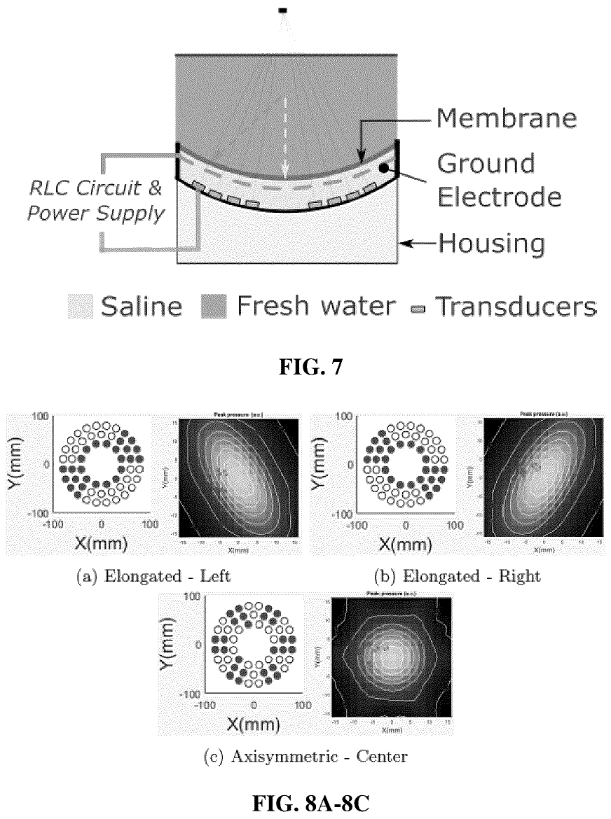

Shock waves produced by a bubble generated by spark deposition in a liquid can be focused to a narrow region. If this can be directed towards a kidney stone, it can help break the stone into small pieces by a combination of stress wave damage and cavitation damage over progressive impacts (FIG. 7). To have a steerable focal region, the use of multiple transducers was proposed. These transducers are arranged on a spherical bowl to enable the waves to focus at the center of this sphere. Each of which can be turned on selectively. The choice of different activated transducer cohorts would lead to different beam shapes at the focal zone, as shown in FIG. 8. These are being proposed to improve stone comminution by aligning them along the typical travel path (as the patient respires) of a stone.

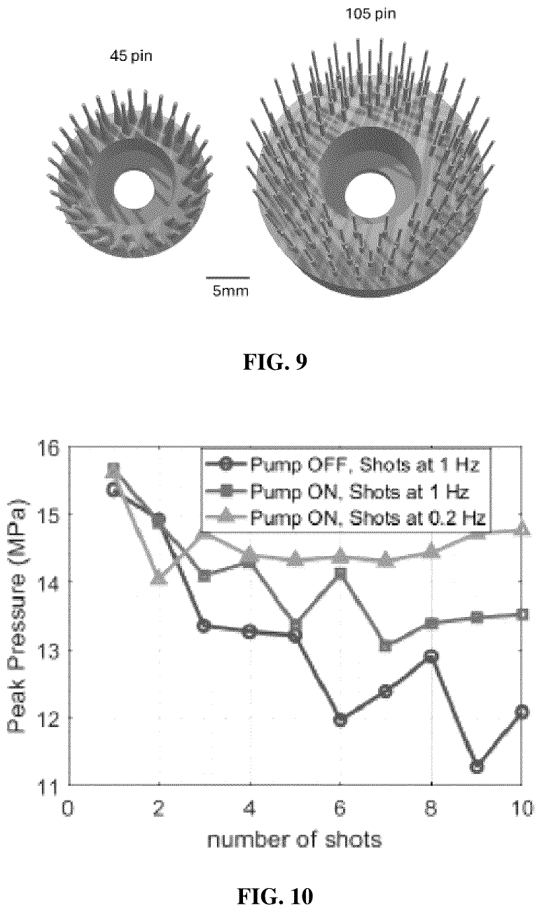

Pin count per transducer: With reference to FIG. 9, increasing the number of pins would mean a reduction in the current density per pin, which should lead to an enhanced pin life.

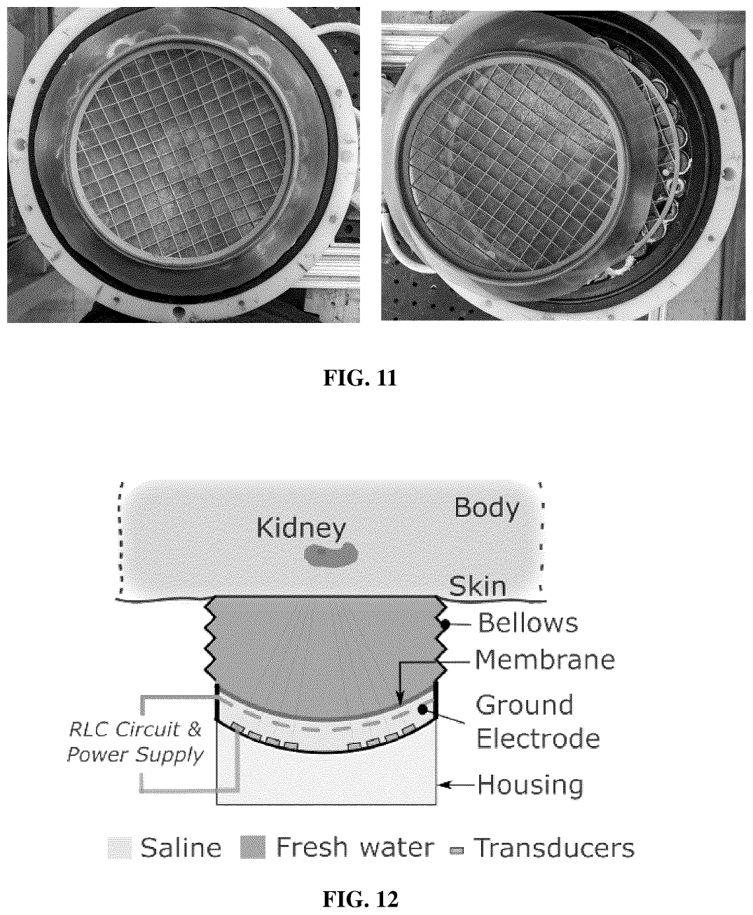

New Housing Unit: A new housing unit has been designed and fabricated to help carry out porcine trials. The design incorporates several features as explained below. FIG. 11 illustrates one embodiment of the shock wave generator.

Ground Electrode: A galvanized iron (GI) 19 AWG wire mesh with a pitch of 0.5″ has been used for these experiments. The GI mesh was manually bent to take the curvature of the bowl. The spacing between the transducer and the mesh may be changed if needed, as this is a critical parameter in the design. The ground electrode could also be replaced quickly, should the need arise (not an expendable). In some embodiments, the ground electrode is a 3D printed stainless steel mesh.

Circulation System: To remove bubbles, continuous degassing has been included. FIG. 10 shows the effect of the saline circulation on the peak pressure at the focal point. By lowering the firing frequency, the pressure reduction can be minimized. In other words, by increasing the flow rate of saline circulation even further, the peak pressure value can be maintained constant, and in some cases provides for higher PRFs.

Silicone Rubber Membrane: Micro-bubbles have a tendency to accumulate at the membrane face. A stiff clear plastic—PETG—was used in conjunction with a solution that can make it difficult for bubbles to attach onto surfaces where it is sprayed. This, however, did not give us satisfactory results. Hence we moved to a different material despite the loss of transparency of the membrane.

Dry Coupling: For our laboratory experiments, we are currently using a freshwater tank to mimic the body. To mimic the clinical conditions, an inflatable bellow would be attached to the housing unit, which will be filled with fresh water, as illustrated in FIG. 12.

Monitoring Devices: To monitor the performance of the machine while stone comminution is ongoing, different sensors—a side-on pressure sensor, salinity and temperature monitor, and an input (transient) current measurement probe, called a Rogowski coil, are being currently added onto the housing unit.

### 4. SYSTEMS AND METHODS

With reference to FIGS. 16-20, a steerable and adjustable focusing electromagnetic or electrohydraulic (SAFE) shock wave generator with multi-unit transducer configurations is illustrated.

A steerable and adjustable focusing electromagnetic (EM) or electrohydraulic (EH) shock wave generator with multi-unit transducer configuration is described to provide flexibility in controlling the beam size and shape in an extracorporeal shock wave lithotripter. Such a device will allow us to better distribute the acoustic shock wave energy to match the anatomic features in the urinary collecting system or respiration movement of the stone to improve stone fragmentation efficiency while reducing tissue injury.

The following challenges were faced. Since the beam's focus is narrow (Ø4-8 mm) and fixed in space, it misses the stone every time it moves out of the pre-set location. This happens 40% of the time, resulting in reduced efficiency. Tracking the stone using an image tracking algorithm yielded poor results in clinical trials. The proposed solution is to broaden the beam at the expense of tissue exposure. An elongated beam addresses the challenges. For example, if the beam shape can be stretched along the respiration direction, it will reduce the chance of missed stones and reduce tissue exposure. In some embodiments, elongation of the beam shape is achieved used different combinations of the total transducer array.

With reference to FIG. 16A-16C, a multi-unit shock wave generator combines an array of individual EM shock units, which can be selectively activated. The transducers can be placed along multiple rows/columns, based on the design of the shock head.

Shape of the pulse and beam steering: With reference to FIG. 17, a system is used to selectively activate different sectors of the shock wave source to create an elongated pressure field in the focal plane. This feature is accomplished by rapid electrical switching between different sectors of transducers, two pairs at a time (green and blue) in contrast to an axisymmetric discharging pattern representative of conventional shock wave lithotripter design (red). By controlling the pair of sectors, we can steer the major axis of the ellipsoidal pressure contour to a particular direction (FIG. 17A). This feature can be used to align the pressure contour of the lithotripter field with the distribution of the stones in the renal collecting system to improve stone comminution efficiency.

For example, the direction of the broader beam size can be aligned either with upper-to-lower pole or pelvis-to ureter direction of the kidney, depending on the geometry and distribution of stones revealed by fluoroscopy. To match with kidney movement due to respiratory motion, the broad beam size can be aligned with the upper-to-lower pole direction (FIG. 17B).

In one embodiment, a shock wave generator includes 66 transducers each with a 18 mm outer diameter and 105 pins. The shock wave generator is controlled to generate various beam sizes, shapes, and pressures.

Lifespan of the transducers. For an electrohydraulic (EH) lithotripter, each transducer comprises of several thousands of pins (the positive electrodes) and one ground wire that is curved to maintain a constant gap between electrodes. The effects of pin number Np on pressure output and electrode degradation are evaluated in order to produce a consistent pressure waveform with increased electrode lifespan. After more than 2,000 pulses, the measured pressure output drops only by 2%, and lifespan of the transducer is expected to exceed at least 10,000 pulses (pressure output drops within 10%).

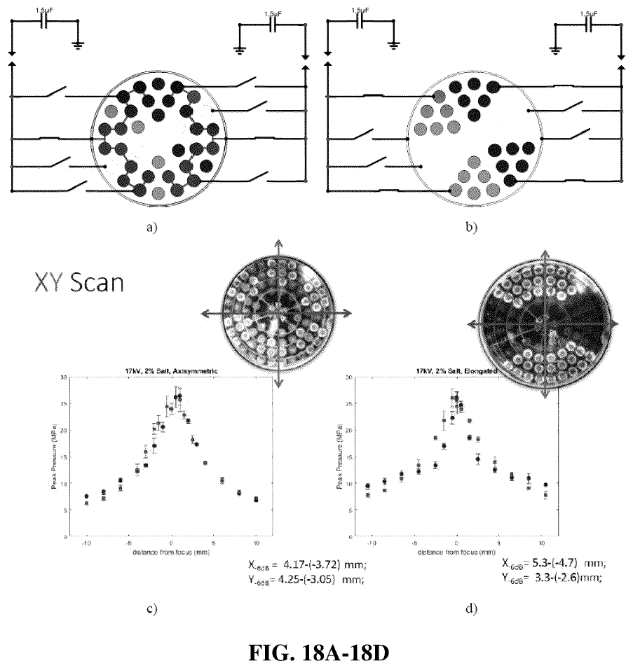

Stone comminution. Experiments were conducted using axisymmetric (several transducers are disconnected in each section) and non-axisymmetric (transducers are disconnected on the two side sections) configurations (FIGS. 18A and 18B). The results show that in axisymmetric case the lithotripter can generate a circular focal zone (FIG. 18C). The focal zone is elongated in the side-to-side direction (red dots in FIG. 18D). Such an elongated focal zone results in stone comminution efficiency much higher than in the axisymmetric case.

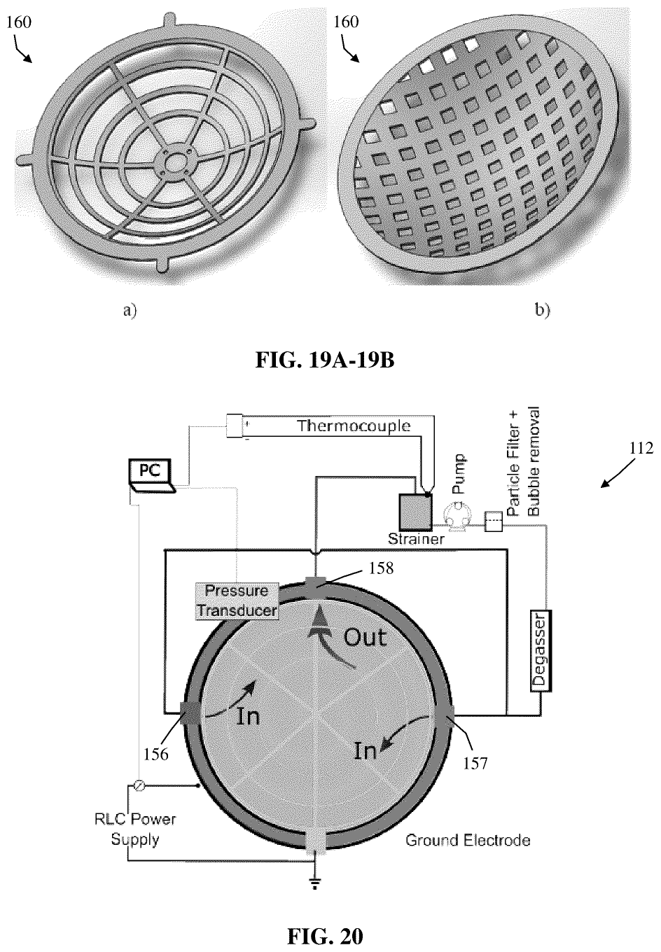

The ground wire. The ground electrode has a larger surface area than the total area of all individual pins in all the positive transducers (FIG. 19). The open area (through which the shock wave passes) is controlled by changing the wire pattern (square, hexagon, trapezia). The ground wire can be shaped to a sphere segment (in the self-focusing transducer) or flat surface (in the transducer with an acoustic lens) to maintain a constant gap between electrodes. In practice, this means that the edge effect is reduced.

Circulation and Coupling. The EH transducer is immersed in a membrane chamber with an electrolyte (saline). Flashing the chamber with electrolyte is critical for acoustic transmission through the membrane wall. The flow velocity V=Q/S, where Q is the pumping flow rate, S is the cross-section area under the membrane (see FIG. 20). Reducing the cross-section would increase the flow speed and detach gas bubbles more effectively. Further, we increase the velocity V by rotating fluid. Two nozzles are placed at 45 degrees in opposition to each other, so that the coolant steering is achieved (FIG. 20). In this case, the fluid velocity has both radial and angular components and thus, is greater than in the case without rotation. Moreover, we may use hydrophilic coating on the internal surfaces in contact with the saline solution to reduce adhesion force between gas bubbles and the acoustically transparent membrane.

In summary, the following is disclosed: a) a Hydrophilic treatment for the ground electrode chamber with a durable coating (such as HydroLAST). The coating repeals gas bubbles from the electrochemical reaction in the working liquid between the pin electrodes and the ground electrode); b) a spherical plastic membrane (such as PETG) to increase the liquid velocity and, hence, the bubble removal capability (by reducing the gap between the pin electrodes and the ground electrode) in the running degassing system; c) an inlet/outlet nozzle design (see FIG. 20) to facilitate rotational flow in-between the pin electrodes and the ground electrode and, those, avoid any stagnant points in the working liquid (such as sodium chloride solution); and a flow circulation chamber with concave, flat, and convex top. Three versions of the membrane provide three different volumes in the chamber with the minimum volume for concave top. When tilted at 30 degrees these membranes also facilitate bubble removal from the chamber by means of gravity force (buoyancy).

In some embodiments, the shock wave generator includes acoustic field feed-back monitoring based on piezoelectric hydrophone measurements. In some embodiments, the shock wave generator includes temperature monitoring. In some embodiments, the shock wave generator includes discharging current monitoring based on Rogowski coil measurements. In some embodiment, the shock wave generator includes increased salt concentration and, hence, offer an increased peak pressure at focus for the EH unit. In some embodiments, a membrane is positioned between the circulated saline chamber and the fresh water chamber.

A new beamforming and beam-steering technology in shock wave lithotripsy is described detailed herein that generates either a circular or preferably an elongated non-circular focal zone that will produce greater stone comminution efficiency with reduced propensity for renal tissue injury. The SAFE shock wave generator can provide a flexible and versatile design to achieve accurate, stable, and safe lithotripsy for kidney stone treatment.

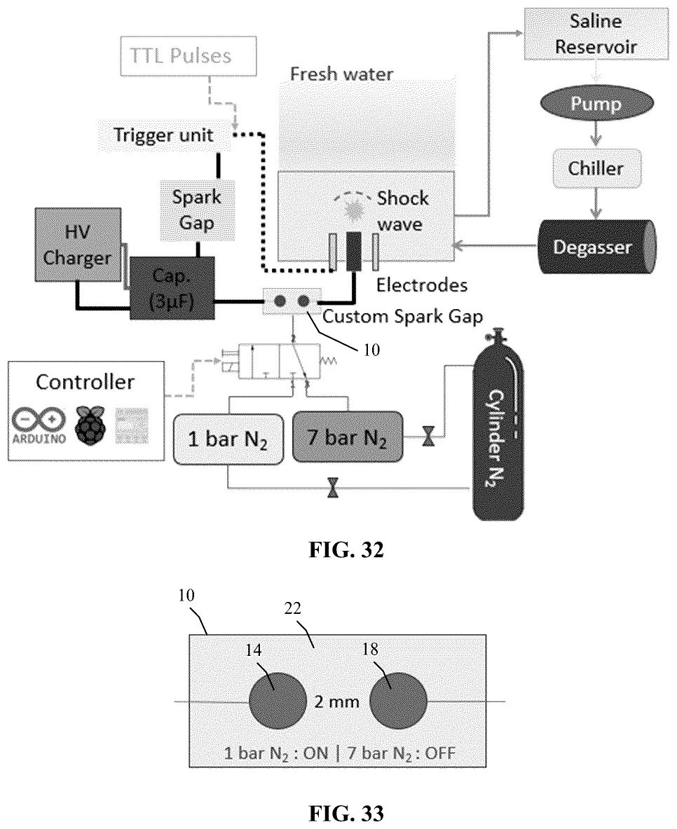

With reference to FIGS. 32 and 33, in some embodiments, the shock wave generator includes individually addressable transducers. In other words, a transducer can be energized or deenergized independent of the other transducers. In some embodiments, the beam generated by the shock wave generator is modified with each transducer being individually addressable, based on feedback from an imaging system (e.g., ultrasound). Individually addressable transducers would enable several beam shapes, sizes, orientations, etc. by selectively activating transducers. In the illustrated embodiment, each of the plurality of transducers is independently energized by the control assembly. In some embodiments, the timing of activation of individual transducers is controlled to generate tandem pulses. In some embodiments, subsets of transducers are energized with a time delay to generate tandem pressure pulses.

In the illustrated embodiment, the control assembly includes a spark gap 10 with a first contact 14, a second contact 18, and a gas 22 positioned between the first contact 14 and the second contact 18. The spark gap 10 is in a closed configuration when the gas is at a first pressure and the spark gap 10 is in an open configuration when the gas is at a second pressure, higher than the first pressure. In some embodiments, the voltage measured between the first contact 14 and the second contact 18 is within a range of approximately 13 kV to approximately 20 kV. In some embodiments, the gas is nitrogen. In some embodiments, the gas is any suitable insulating gas such as N2 or SF6. In some embodiments, the first pressure is approximately 1 bar and the second pressure is approximately 7 bar. In some embodiments, the first pressure and the second pressure are selected from the Paschen's curve for the gas. In other embodiments, the control assembly includes a plurality of solid-state transducers (or switches of any type for electrical connection that can be quickly made and broken) for individual control and activation of each of the plurality of transducers.

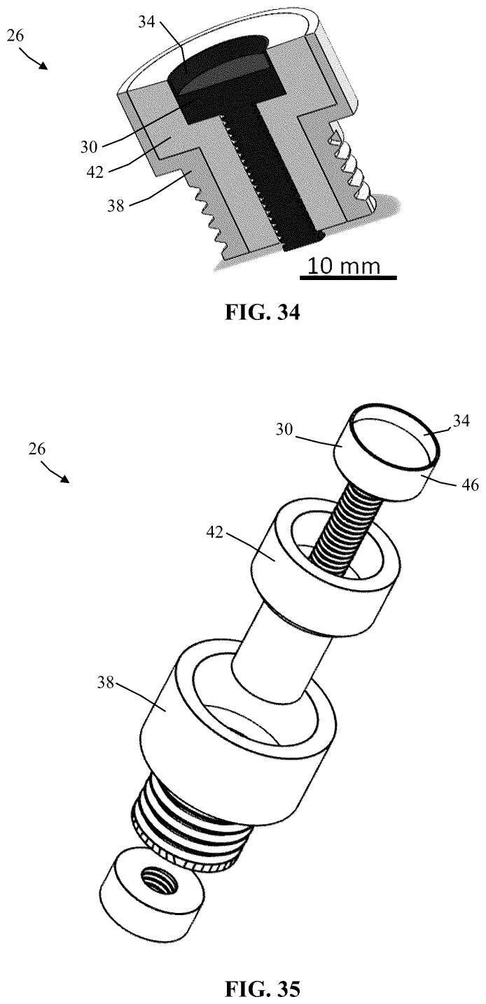

With reference to FIGS. 34 and 35, a transducer 26 is illustrated and may comprise one or more of the plurality of transducers positioned on the base of the shock wave generator. The transducer 26 includes an electrode 30 with a ring 34, a ground electrode 38, and an insulator 42 positioned between the electrode 30 and the ground electrode 38. The ring 34 forms at least part of an outer circumferential surface 46 of the electrode 30. Advantageously, the electrode 30 is manufactured by machining. The electrode 30 also advantageously has greater surface area that is easier to cool.

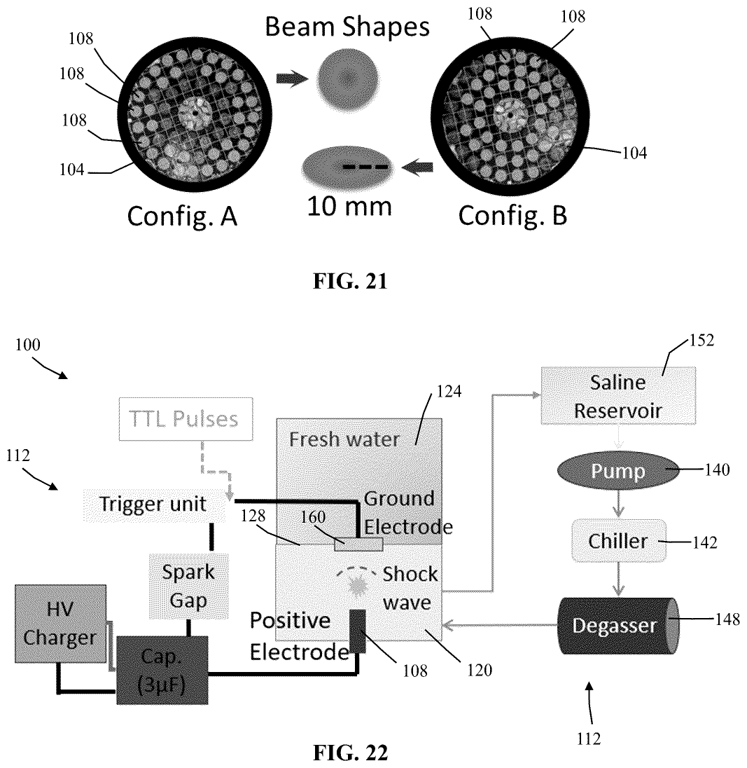

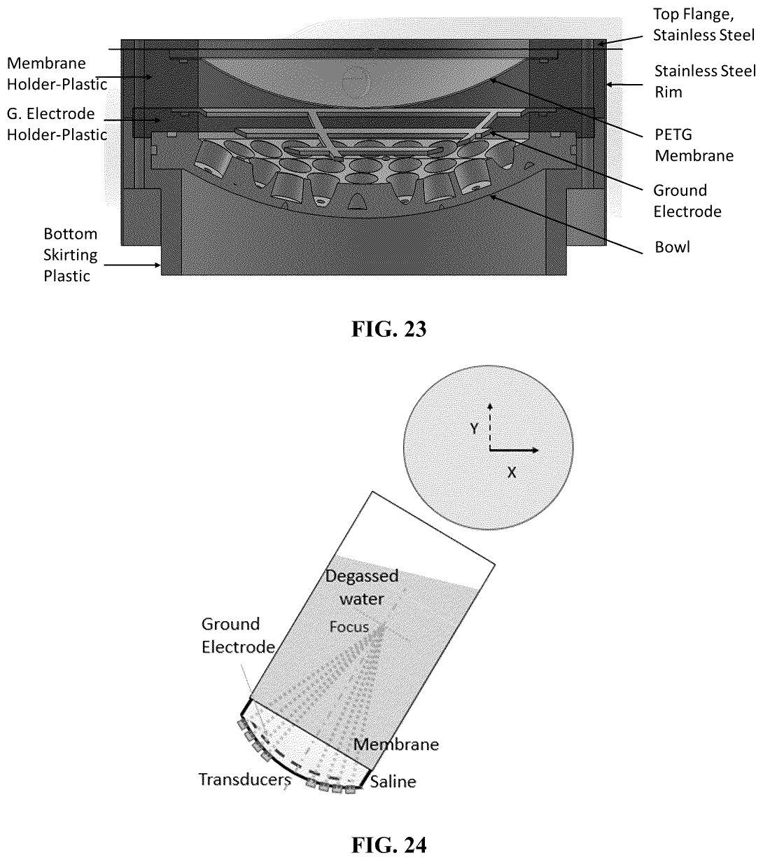

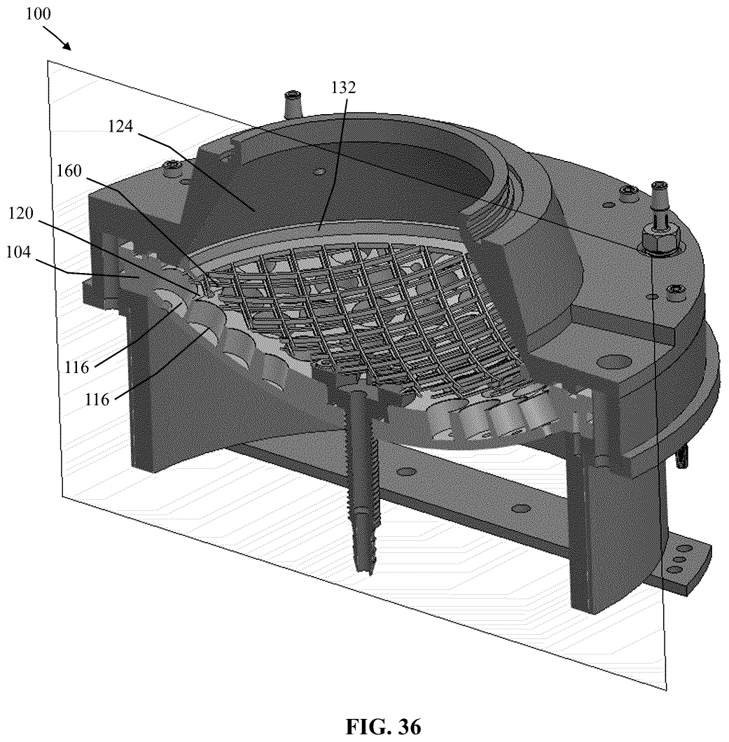

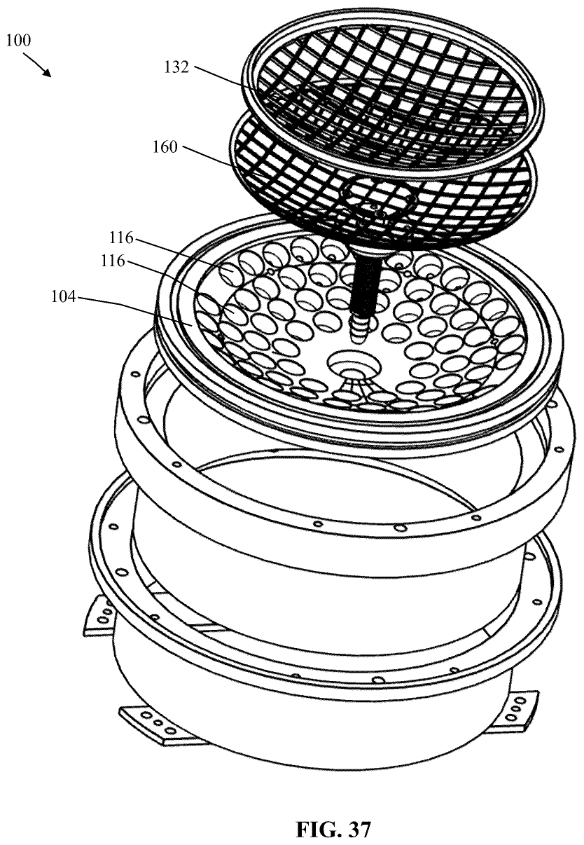

With reference to FIGS. 21, 22, and 36 and 37, a shock wave generator 100 includes a base 104, a plurality of transducers 108 positioned on the base 104, and a control assembly 112 electrically coupled to the plurality of transducers 108. In some embodiments, the base is spherical and includes a plurality of bores 116 (FIGS. 36 and 37) that receive the plurality of transducers. In some embodiments, the base is planar.

A first portion of the plurality of transducers are energized by the control assembly in a first configuration (Config. A, FIG. 21) to generate a first beam shape; and a second portion of the plurality of transducers are energized by the control assembly in a second configuration (Config. B, FIG. 21) to generate a second beam shape. In the illustrated embodiment, the first beam shape is circular, and the second beam shape is elongated. In some embodiments, a first portion of the plurality of transducers are energized by the control assembly in a first configuration to generate a first beam size; and a second portion of the plurality of transducers are energized by the control assembly in a second configuration to generate a second beam size. In some embodiments, a third portion of the plurality of transducers are energized in coordination with the adjustment in energization and inactivation of the first and second portions of the plurality of the transducers by the control assembly to change either the size of the circular beam or the shape, size and orientation of the elongated beam. In some embodiments, a first portion of the plurality of transducers are energized by the control assembly in a first configuration to generate a first beam shape in a first orientation; and a second portion of the plurality of transducers are energized by the control assembly in a second configuration to generate the first beam shape in a second orientation.

In some embodiments, a first portion of the plurality of transducers are energized by the control assembly in a first configuration to generate a first beam shape with a first pressure distribution; and a second portion of the plurality of transducers are energized by the control assembly in a second configuration to generate the first beam shape with a second pressure distribution. In other words, the damage zone of the beam shape can be moved and controlled based on the energization of the plurality of transducers.

In some embodiments, the plurality of transducers comprises at least 30 transducers. In some embodiments, the plurality of transducers comprises at least 66 transducers. In some embodiments, one or more of the transducers of the plurality of transducers includes a plurality of pins (FIG. 9). In some embodiments, the plurality of pins comprises at least 45 pins. In some embodiments, the plurality of pins comprises at least 105 pins. In some embodiments, one or more of the transducers of the plurality of transducers includes an electrode with a ring, a ground electrode, and an insulator positioned between the electrode and the ground electrode (FIGS. 34 and 35).

With continued reference to FIGS. 22 and 36, the shock wave generator 100 includes a first chamber 120 with a first fluid. The first chamber is at least partially defined by the base. The shock wave generator further includes a second chamber 124 with a second fluid. In some embodiments, the second chamber is at least partially defined by a bellow. In some embodiments, the first fluid is saline. In some embodiments, the second fluid is water.

In the illustrated embodiment, the shock wave generator 100 includes a membrane 128 positioned between the first chamber 120 and the second chamber 124. In some embodiments, a holder 132 supports the membrane. In some embodiments, the membrane is a spherical plastic membrane with a hydrophilic coating. In some embodiment, the membrane is any suitable elastic material that does not drastically attenuate the shock waves.

With continued reference to FIG. 22, the shock wave generator includes a circulation assembly 136 fluidly coupled to the first chamber 120. The circulation assembly 136 includes a pump 140 that circulates the first fluid, a chiller 144 that controls the temperature of the first fluid, and a degasser 148 that removes bubbles from the first fluid. In some embodiments, the shock wave generator further includes a reservoir 152 with the first fluid in fluid communication with the first chamber. In some embodiments, the shock wave generator includes means to adjust or control the conductivity in the first fluid.

With reference to FIG. 20, in some embodiments, the circulation assembly includes a first inlet 156, a second inlet 157, and a first outlet 158 fluidly coupled to the first chamber. In some embodiments, the first inlet is positioned opposite the second inlet. The first inlet and the second inlet direct the first fluid into the first chamber with a radial and circumferential direction. In other embodiments, multiple outlets are provided to aid in balancing the pressure inside the first chamber.

In some embodiments, the shock wave generator includes a ground electrode 160 positioned in the first chamber 120. In some embodiments, the ground electrode is spherical shaped and includes a grid pattern with a plurality of openings. In some embodiments, the ground electrode is shaped to match the curvature of the bowl.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

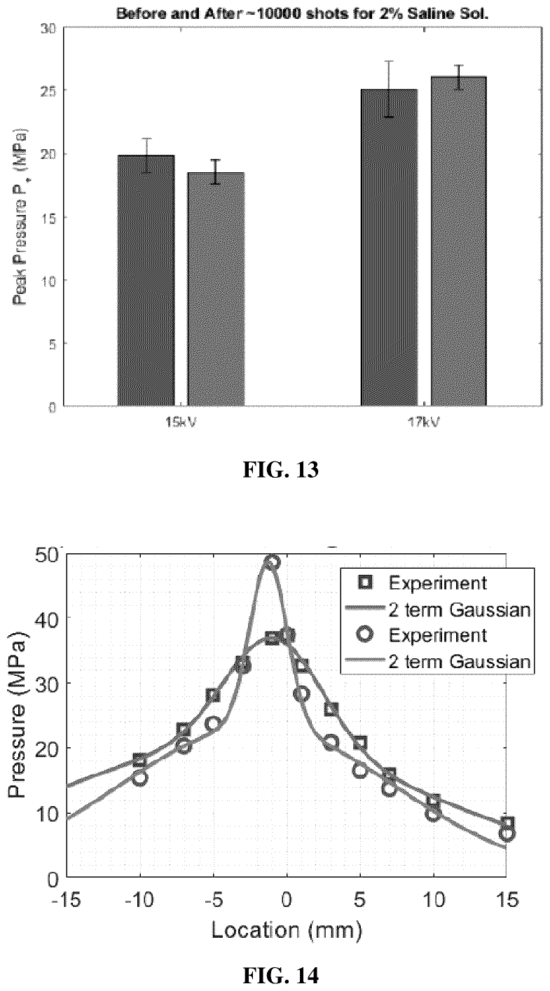

Pin Life: With reference to FIGS. 13 and 14, the peak pressure measured at the focus is a good representation of the state of the pins. To get an initial idea on how these pins are performing, pressure values were measured at the focal point before and after a series of stone comminution experiments. This amounted to about 10,000 shots. The peak pressure as remained unchanged within statistical error. This needs to be compared with a typical life of 1000-2000 shots for the single electrodes typically used in electrohydraulic lithotripters.

Pressure Scan: A preliminary scan of the pressure on the focal plane was carried out for the new housing unit (FIG. 14) and the peak pressures and the −6 dB focal region have been tabulated in Tables 3 and 4.

TABLE 3
Peak pressures for different configurations compared. NHU:
New Housing Unit, Different power supply and housing unit,

Input
Experiments

| Bowl Configuration | Axi. beam | Elo. beam |
|---|---|---|
| 66 × 45 pin, 1%, 15 kV¹ | 21.8 MPa | 21.1 MPa |
| 42 × 105 pin, 2%, 17 kV² | 23.3 MPa | 26.9 MPa |
| 66 × 45 pin, 1%, 15 kV, NHU | | 42 MPa |
| 36 × 105 pin, 1%, 15 kV, NHU* | | 48.95 MPa |

*NHU, ground electrode spacing lowered by 50% to 12.5 mm.

TABLE 4
Beam widths for different configurations compared. NHU:
New Housing Unit, Different power supply and housing unit

| | Input | | | | | |
|---|---|---|---|---|---|---|
| | Experiments | | | | Numerical | |
| Bowl Configuration | Axi. beam | | Elo. beam | | Axi. beam | Elo. beam |
| 66 × 45 pin, 1%, 15 kV¹ | 14.8 × 14.8 | mm | 15.1 × 22.7 | mm | 13 × 13 mm | 13 × 18.5 mm |
| 42 × 105 pin, 2%, 17 kV | 7.89 × 7.30 | mm² | 10 × 5.9 | mm² | | |
| 66 × 45 pin, 1%, 15 kV, NHU | | | 10 × 6.4 | mm | | |
| 36 × 105 pin, 1%, 15 kV, NHU | | | 15.6 × 5.8 | mm | | |

The new housing unit provides a higher pressure than the previous unit, but the −6 dB focal region is narrower.

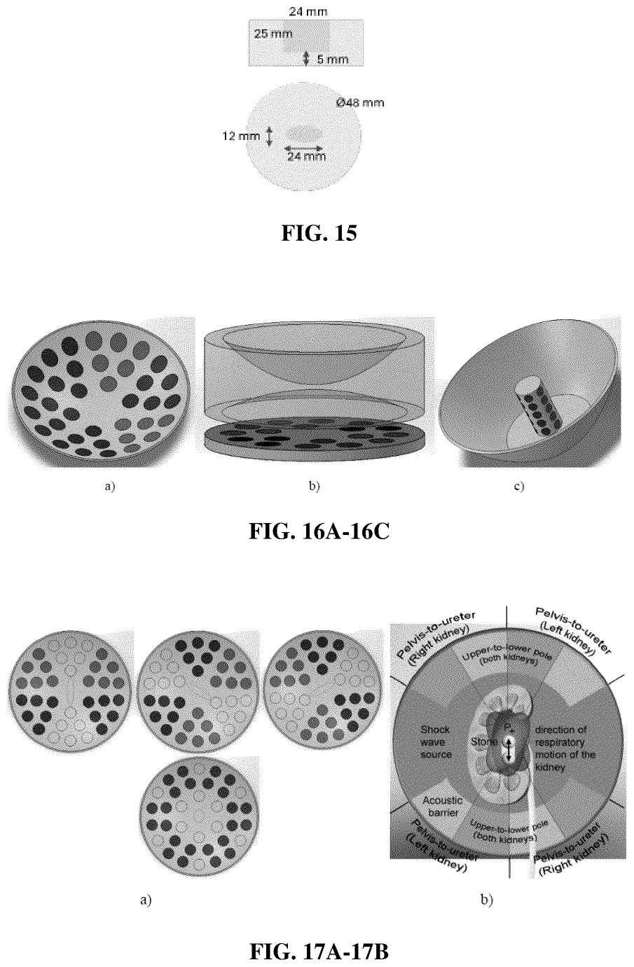

Stone Comminution Results: Stone comminution was carried out using a holder that was made out of ballistic gel (FIG. 15), a material that can mimic the acoustic and mechanical properties of the human body. This holder was placed within the freshwater tank attached to the shock head (FIG. 7). The holder was filled with tap water and placed at a suitable location in the vicinity of the focal plane. Table 5 details results comparison.

TABLE 5
Stone comminution results after 150 shots at 0.1 Hz using 17 kV and 1% saline for
different cases. All are for an elongated beam. (Sankin et. al 2023), at 20 Kv.

| | Focal (PU) | Post-Focal (SH1) | Pre-Focal (SH2) |
|---|---|---|---|
| | SC 2 mm | | |
| 45 pin, 66 transducer, NHU | 35.37 ± 5.57 | 23.88 ± 7.16 | 30.71 ± 2.95 |
| 105 pin, 42 transducer, NHU | 21.07 ± 2.67 | — | 40.40* |
| 105 pin, 36 transducer, NHU | 38.5 ± 4.1 | — | — |
| 45 pin, 66 transducer¹ | 42.2 ± 3.5 | — | — |
| | SC 2.8 mm | | |
| 45 pin, 66 transducer, NHU | 60.96 ± 9.68 | 39.73 ± 10.64 | 48.41 ± 2.87 |
| 105 pin, 42 transducer, NHU | 44.23 ± 11.16 | — | 58.20* |
| 105 pin, 36 transducer, NHU | 86 ± 3.06 | — | — |
| 45 pin, 66 transducer¹ | 80 ± 9 | — | — |

The stone comminution results for the new housing unit with the 105 pin-transducer case almost comparable to the previous data. The previous experiments were conducted at 20 kV, whereas the current experiments are at 17 kV, showing an improvement even in terms of the power used by the Lithotripter.

### Example 2

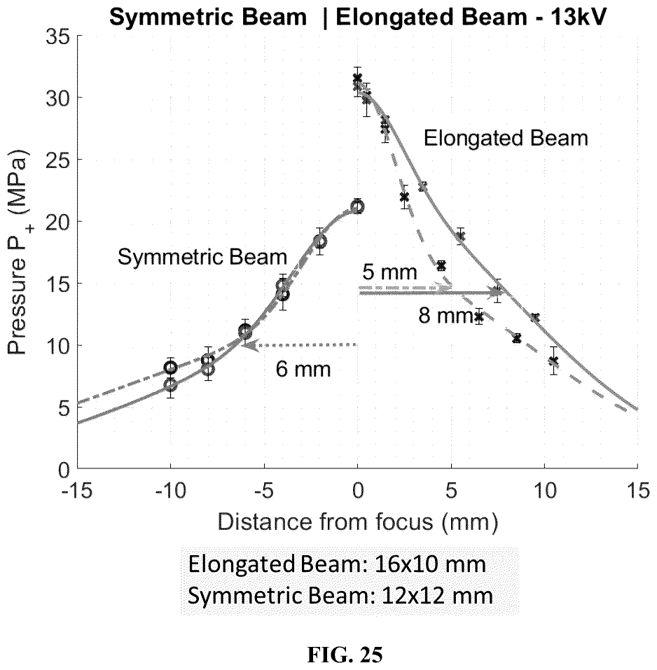

Pressure Testing. With reference to FIGS. 24 and 25, pressure testing demonstrates results measured for a symmetric beam and an elongated beam generated by a shockwave generator/

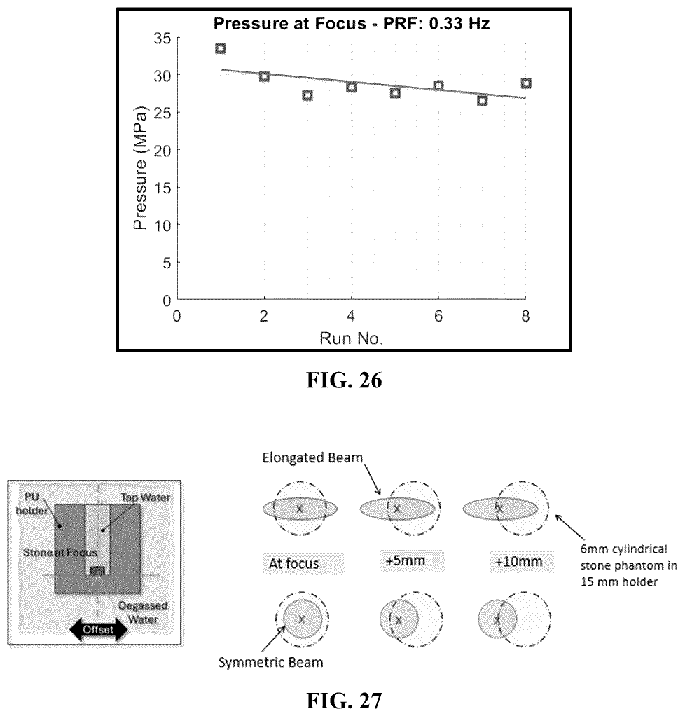

Saline Circulation. With reference to FIG. 26, saline flow is monitored and mainted at, or greater than 6 lpm to ensure that the pressure does not drop at the focal point. The saline is temperature controlled to ensure heat is effectively removed during the spark generation activity, for example. The degassing system is incorporated into the circulatory loop to ensure that bubbles are no longer in the saline, since bubbles can attenuate the shock wave generated. In the illustrated example, a steady pressure output at PFR of 0.3 Hz is demonstrated.

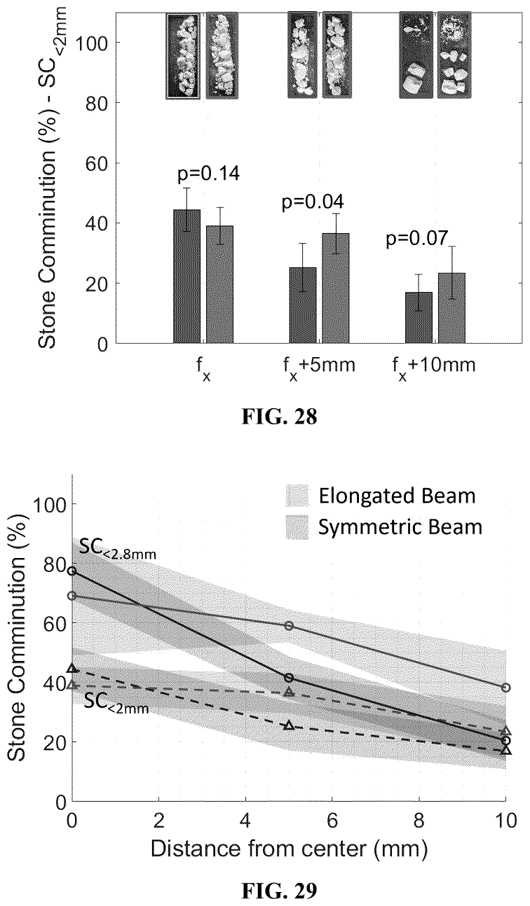

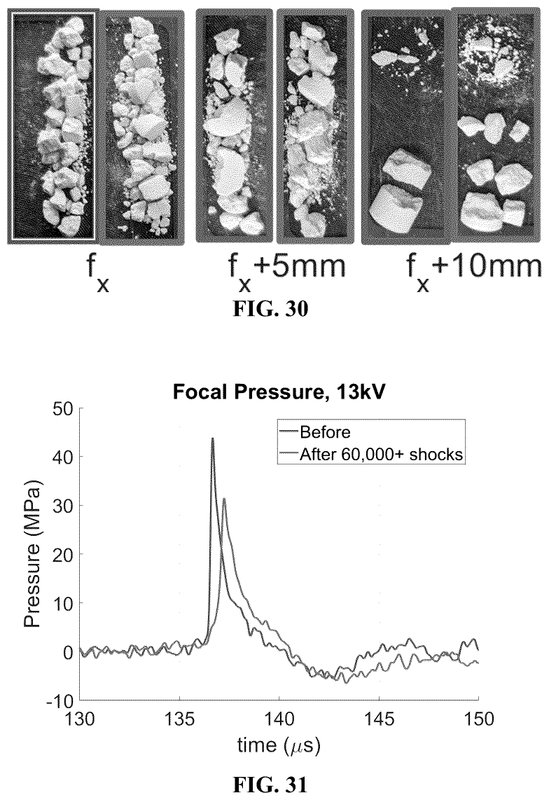

Stone Comminution. With reference to FIGS. 27, 28, 29, and 30, a stone comminution study is performed to mimic respiration. In some embodiments, a Begostone® (available from BEGO of Lincoln, RI) phantom is utilized to mimic the mechanical properties of a kidney stone. In the illustrated example, cylindrical stones with a 6 mm outer diameter and 6 mm length were utilized.

Lifespan. With reference to FIG. 31, a lifespan of the design is tested with pins that had not degraded after 10,000 pulses. After 60,000 pulses, the focal pressure drop was approximately 30%. Comparatively, for a 25% drop in pressure, the conventional standard in electrohydraulic lithotripter delivers only 2000 pulses, and the electroconductive (ECL) technology delivers approximately 6000 pulses.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A shock wave generator comprising:
a base;
a plurality of transducers positioned on the base;
a control assembly electrically coupled to the plurality of transducers;
a first chamber with a first fluid; wherein the first chamber is at least partially defined by the base;
a second chamber with a second fluid;
a membrane positioned between the first chamber and the second chamber; and
a circulation assembly fluidly coupled to the first chamber; wherein the circulation assembly includes a pump that circulates the first fluid, a chiller that controls the temperature of the first fluid, and a degasser that removes bubbles from the first fluid.

2. The shock wave generator of claim 1, wherein a first portion of the plurality of transducers are energized by the control assembly in a first configuration to generate a first beam shape; and a second portion of the plurality of transducers are energized by the control assembly in a second configuration to generate a second beam shape.

3. The shock wave generator of claim 2, wherein the first beam shape is circular, and the second beam shape is elongated.

4. The shock wave generator of claim 2, wherein a third portion of the plurality of transducers are energized in coordination with the adjustment in energization and inactivation of the first and second portions of the plurality of the transducers by the control assembly to change either the size of the circular beam or the shape, size and orientation of the elongated beam.

5. The shock wave generator of claim 1, wherein a first portion of the plurality of transducers are energized by the control assembly in a first configuration to generate a first beam size; and a second portion of the plurality of transducers are energized by the control assembly in a second configuration to generate a second beam size.

6. The shock wave generator of claim 1, wherein a first portion of the plurality of transducers are energized by the control assembly in a first configuration to generate a first beam shape in a first orientation; and a second portion of the plurality of transducers are energized by the control assembly in a second configuration to generate the first beam shape in a second orientation.

7. The shock wave generator of claim 1, wherein a first portion of the plurality of transducers are energized by the control assembly in a first configuration to generate a first beam shape with a first pressure distribution; and a second portion of the plurality of transducers are energized by the control assembly in a second configuration to generate the first beam shape with a second pressure distribution.

8. The shock wave generator of claim 1, wherein each of the plurality of transducers is independently energized by the control assembly.

9. The shock wave generator of claim 7, wherein the control assembly includes a spark gap with a first contact, a second contact, and a gas positioned between the first contact and the second contact; wherein the spark gap is in a closed configuration when the gas is at a first pressure and the spark gap is in an open configuration when the gas is at a second pressure, higher than the first pressure.

10. The shock wave generator of claim 9, wherein the gas is nitrogen and the first pressure is 1 bar and the second pressure is 7 bar.

11. The shock wave generator of claim 8, wherein the control assembly includes a plurality of solid-state transducers.

12. The shock wave generator of claim 1, wherein the plurality of transducers comprises at least 30 transducers.

13. The shock wave generator of claim 1, wherein a first transducer of the plurality of transducers includes a plurality of pins; wherein the plurality of pins comprises at least 105 pins.

14. The shock wave generator of claim 13, further comprising a ground electrode positioned in the first chamber; wherein the ground electrode is spherical shape and includes a grid pattern with a plurality of openings.

15. The shock wave generator of claim 1, wherein a first transducer of the plurality of transducers includes an electrode with a ring, a ground electrode, and an insulator positioned between the electrode and the ground electrode; wherein the ring forms at least part of an outer circumferential surface of the electrode.

16. The shock wave generator of claim 1, further comprising a reservoir with the first fluid in fluid communication with the first chamber; and wherein the second chamber is at least partially defined by a bellow.

17. The shock wave generator of claim 1, wherein the membrane is a spherical plastic membrane with a hydrophilic coating.

18. The shock wave generator of claim 1, wherein the first fluid is saline and the second fluid is water.

19. The shock wave generator of claim 1, wherein the base is spherical and includes a plurality of bores that receive the plurality of transducers.

20. The shock wave generator of claim 1, wherein the circulation assembly includes a first inlet, a second inlet, and first outlet fluidly coupled to the first chamber; wherein the first inlet is positioned opposite the second inlet; and wherein the first inlet and the second inlet direct the first fluid into the first chamber with a radial and circumferential direction.
